(19) United States
(12) Patent Application Publication
(10) Pub. No.: US 2024/0252578 A1
(43) Pub. Date: Aug. 1, 2024

(54) DIETARY SUPPLEMENT

(71) Applicant: Shawnkya Turner, Milwaukee, WI (US)

(72) Inventor: Shawnkya Turner, Milwaukee, WI (US)

(21) Appl. No.: 18/412,513

(22) Filed: Jan. 13, 2024

Related U.S. Application Data
(60) Provisional application No. 63/442,568, filed on Feb. 1, 2023.

(51) Int. Cl. A61K 36/906; A61K 31/59; A61K 33/06; A61K 36/54; A61K 36/63; A61K 36/88

(52) U.S. Cl. CPC A61K 36/906; A61K 31/59; A61K 33/06; A61K 36/54; A61K 36/63; A61K 36/88

(57) ABSTRACT

A dietary supplement made from a combination of ingredients. In particular, the combination includes, as active ingredients, therapeutically effective amounts of vitamin D, calcium, ginger root powder, olive leaf powder, cinnamon bark powder, and garlic bulb extract in specific ranges of proportions as outlined herein. The dietary supplement is provided as a capsule filled with a mixture of the active ingredients and suitable excipients, such as rice flour and vegetable magnesium stearate.

### CROSS REFERENCE TO RELATED APPLICATIONS

This application claims the priority benefit of U.S. provisional patent application No. 63/442,568, filed Feb. 1, 2023 and entitled DIETARY SUPPLEMENT, the entire contents of which are hereby incorporated by reference herein for all purposes.

### FIELD OF THE DISCLOSURE

The present invention generally relates to a dietary supplement. More particularly, it relates to a dietary supplement with antioxidant properties for immune system health and wellness and a method for improving and maintaining immune system health and wellness by taking an effective amount of the dietary supplement daily.

### DESCRIPTION OF BACKGROUND

Numerous natural substances have been found to possess antioxidant properties, and numerous antioxidant dietary supplement compositions have been developed for immune system health and wellness.

However, the prior art antioxidant dietary supplement compositions and methods of making and/or taking the same fail to consider the optimized dietary supplement composition and proportions of ingredients to achieve optimal results. The proper composition of ingredients is critically important for the intended functions, such as long-term improvement and maintenance of immunity to disease and wellness. In addition, the proportion of the different ingredients of a particular antioxidant dietary supplement composition is critical for the intended long-term benefits. Accordingly, there is a need for an antioxidant dietary supplement of optimized selection and proportions of ingredients for improving and maintaining the immune system health and wellness of consumers of the dietary supplement composition.

### SUMMARY OF THE DISCLOSURE

Generally speaking, pursuant to the various embodiments, the present disclosure provides a dietary supplement with antioxidant properties for immune system health and wellness, and methods for improving and/or maintaining immune system health and wellness by consuming an effective amount of the dietary supplement daily. In accordance with the present disclosure, formulas of the dietary supplement each include an optimized collection of ingredients and desired corresponding proportions. In one implementation, an amount of the dietary supplement to be taken daily includes 50 mcg vitamin D as cholecalciferol, 600 mg calcium as calcium carbonate, 750 mg of ginger root powder, 500 mg of olive leaf powder, 500 mg of cinnamon bark powder, and 300 mg of garlic bulb extract.

### DETAILED DESCRIPTION

Various dietary substances and chemical compounds have been found to have long-term benefits for immunity and wellness. As used herein, without limitation, a dietary substance or chemical compound can be a chemical composition, a plant extract, an herb, a mineral, a vitamin, a phytonutrient, an amino acid or an enzyme. The same can also be referred to as an ingredient when used in combination to form a dietary supplement composition. Ingredients of the dietary supplement of this disclosure include therapeutically effective amounts of vitamin D (as cholecalciferol), calcium (as calcium carbonate), ginger root powder, olive leaf powder, cinnamon bark powder, and garlic bulb extract. Each ingredient is described below, followed by illustrative examples of formulas with optimized proportions of these ingredients.

### Vitamin D (as Cholecalciferol):

It is well known that vitamin D enhances the intestinal absorption of dietary calcium and phosphorus. In addition, vitamin D is a natural antioxidant. Vitamin D deficiency has been linked to major chronic diseases associated with oxidative stress, inflammation, and aging, including cardiovascular and neurodegenerative diseases, diabetes, and cancer. It has been proposed that pharmacological interventions targeting either vitamin D deficiency or its key downstream effects, including defective autophagy and abnormal pro-oxidant and pro-inflammatory responses, may be able to limit the onset and severity of major cerebrovascular diseases, such as stroke and cerebrovascular malformations, which are leading causes of disability and death all over the world. In addition, Vitamin D is believed to play an important role in regulating and controlling the immune system response. Vitamin D is provided in dietary supplements according to this disclosure as cholecalciferol, having the molecular formula C₂₇H₄₄O.

### Calcium (as Calcium Carbonate):

In addition to its popularly known benefits for teeth and bone health, calcium also supports immune health. In particular, calcium signaling (by way of the calcium ion Ca2+) is known to be essential to immunity. Regulated increases in cytosolic and organellar Ca2+ concentrations in lymphocytes control complex and crucial effector functions such as metabolism, proliferation, differentiation, antibody and cytokine secretion and cytotoxicity. Altered Ca2+ regulation in lymphocytes leads to various autoimmune, inflammatory and immunodeficiency syndromes. Several types of plasma membrane and organellar Ca2+-permeable channels are functional in T cells, fine-tuning T cell signaling to match complex immune responses, thereby shaping immunity and the pathogenesis of immune-mediated diseases. Calcium is provided in dietary supplements according to this disclosure as calcium carbonate, having the molecular formula CaCO₃.

### Ginger Root Powder:

The health benefits of ginger are mainly attributed to its phenolic compounds, such as gingerols (for example 6-gingerol, molecular formula C₁₇H₂₆O₄) and shogaols (for example 6-shogaol, molecular formula C₁₇H₂₄O₃). Accumulated investigations have demonstrated that ginger possesses multiple biological activities, including antioxidant, anti-inflammatory, antimicrobial, anticancer, neuroprotective, cardiovascular protective, respiratory protective, antiobesity, antidiabetic, antinausea, and antiemetic activities.

Ginger root powder is commonly standardized by gingerol and/or shogaol content, for example, to 5%, 10%, or 20% gingerols and/or shogaols. A suitable ginger root powder for use in dietary supplements according to this disclosure has 20% gingerols and/or shogaols.

### Olive Leaf Powder:

Olive leaves contain a substance called oleuropein (molecular formula C₂₅H₃₂O₁₃), which has many known and possible human health benefits, including reduced cholesterol, reduced inflammation, diabetic health benefits, antimicrobial benefits, anti-HIV benefits, and anticancer effects with possible efficacy in humans.

Cholesterol reduction: Studies on hypercholesterolemic rats suggest that olive leaf extract (OLE) is more effective than pure oleuropein at lowering cholesterol, suggesting synergy between oleuropein and another substance in the leaf.

Diabetic health benefits: OLE has been found to reduce glucose levels in the blood. Hypoglycemic activities of olive leaf are attributed to two mechanisms: potentiation of glucose-induced insulin release and increased peripheral uptake of glucose. It has also been found that OLE may prevent diabetic neuropathy by reducing glucose-induced apoptosis through the inhibition of neural caspase 3 activation.

Antimicrobial benefits: Laboratory studies found that OLE can kill a number of microorganisms including yeasts, bacteria, and fungi. Oleuropein is converted into elenolic acid (molecular formula C₁₁H₁₄O₆) in the body, which may prevent viruses and bacteria from replicating, thus promoting immune health. In vitro studies show that OLE has antimicrobial activities against E. coli, Pseudomonas aeruginosa, S. aureus, K. pneumoniae, Trichophyton mentagrophytes, Microsporum canis, T. rubrum, and Candida albicans.

Anti-HIV benefits: Anti-HIV properties of OLE include upregulation of the expression of apoptosis inhibitor proteins as well as protein kinase signaling molecules.

Potential anticancer benefits: OLE promotes cell differentiation and induces DNA fragmentation leading to apoptosis in leukemia cell line.

Olive leaf powder is standardized for oleuropein content. A suitable olive leaf powder for use in dietary supplements according to this disclosure can, for example, have 12% or 20% oleuropein.

Cinnamon (Cinnamomum cassia) Bark Powder:

Nutritive compounds of cinnamon (Cinnamomum cassia) with known or potential health benefits include cinnamic acid (molecular formula C₉H₈O₂) and various flavonoids, the flavonoids including cinnamaldehyde (molecular formula C₉H₈O), catechins and epicatechins (both flavan-3-ols, or flavanols, and isomers of the molecular formula C₁₅H₁₄O₆) as well as procyanidin B2 (a proanthocyanidin consisting of two bonded molecules of (−)-epicatechin); gossypin (molecular formula C₂₁H₂₀O₁₃), gnaphalin (molecular formula C₂₀H₂₄O₆), hesperidin (molecular formula C₂₈H₃₄O₁₅), hibifolin (molecular formula C₂₁H₁₈O₁₄), hypolaetin (molecular formula C₁₅H₁₀O₇), oroxindin (molecular formula C₂₂H₂₀O₁₁), and quercetin (molecular formula C₁₅H₁₀O₇). These compounds are known or believed to have a number of health benefits, including diabetic health benefits, limiting Alzheimer-induced changes in the brain and brain insulin signaling, anti-microbial activity (by the cinnamaldehyde component), cardio protective and associated anti-inflammatory benefits of cinnamaldehyde and cinnamic acid, lipid lowering effects, impeded activity of melanoma cells, reduced blood pressure, and antioxidant activity (decreased markers of oxidative stress).

Cinnamon (Cinnamomum cassia) bark powder is standardized by flavonoid content. Suitable cinnamon bark powder for use in dietary supplements according to this disclosure has 8% flavonoids.

### Garlic Bulb Extract:

Garlic (Allium sativum L.) contains sulfur compounds that are formed when a fresh garlic clove is chopped, crushed, or chewed, which scientists have determined to be the source of most of garlic's health benefits. Of these, the compound allicin (molecular formula C₆H₁₀OS₂) is perhaps the best known for its nutritional benefits.

Garlic bulb extracts are commonly standardized by their allicin content, such as 0.6% or 1.1-1.3% allicin. A suitable garlic bulb extract for use in dietary supplements according to this disclosure contains 0.2%-1% allicin. Garlic is also rich in vitamin C, vitamin B6, and manganese.

In human studies, garlic supplements have been found to have a significant impact on reducing blood pressure in people with initially high blood pressure (hypertension). Human garlic consumption can also lower total and LDL cholesterol levels.

In accordance with the present disclosure, illustrative embodiments of the antioxidant dietary supplement are set forth in the formulas below for making the optimal dietary supplement compositions for improving and maintaining human and animal immunity and wellness.

### Formulas

In the following formula tables, the right column indicates the percentage by mass of each active ingredient within the total active ingredient mass of the dietary supplement composition of the respective formula. The middle column indicates the amount by mass (expressed in metric mass units) of a daily serving of each active ingredient. The bottom row indicates the total active ingredient mass of a daily serving of the dietary supplement composition. Daily serving amounts for the respective ingredients vary from formula to formula within the following ranges: Vitamin D (as cholecalciferol): 0.015-0.100 mg (15-100 mcg), equal to 600-4,000 IU (international units); calcium (as calcium carbonate): 500-600 mg; ginger root powder: 500-850 mg; olive leaf powder: 500-750 mg; cinnamon bark powder: 500-750 mg; garlic bulb extract: 300-550 mg. In embodiments, the dietary supplement composition consists essentially of the formula of active ingredients but can also contain one or more suitable excipients contained in the capsules for one or more purposes such as binding, thickening, bulking, anticaking, lubrication, release, antifoaming, appearance enhancement, bioavailability improvement, clumping prevention, and promoting flow and/or mixing of the active ingredients. Such suitable excipients may include, without limitation, rice flour and a stearic acid salt such as magnesium stearate (which may be from a vegetable source). In addition, it will be understood (in accordance with a common labeling convention for nutrient minerals) that the mass of calcium refers to that of elemental calcium only and not the entire mass of a calcium salt containing the calcium of the composition. “Total active ingredient mass” will be understood to exclude the mass of any such excipients and the mass of the anion component of the calcium salt. Finally, while forms of Vitamin D and calcium are specified in the tables as cholecalciferol and calcium carbonate, it will be understood that another form of Vitamin D can be provided in equivalent mass amounts containing the same IU amounts of Vitamin D (where cholecalciferol contains 40 IU of Vitamin D per mcg), and that calcium can be provided as another suitable calcium salt (such as calcium citrate) in amounts containing the same masses of elemental calcium as specified in the tables.

Formula 1:

| Ingredient | Amount (mg) | % of Total Comp |
|---|---|---|
| Vitamin D (as | 0.05 | 0.002% |
| cholecalciferol) | | |
| Calcium (as calcium | 600 | 22.641% |
| carbonate CaCO₃) | | |
| Ginger Root Powder | 750 | 28.301% |
| Olive Leaf Powder | 500 | 18.868% |
| Cinnamon Bark Powder | 500 | 18.868% |
| Garlic Bulb Extract | 300 | 11.321% |
| Total: | 2650.05 | 100.000% |

Formula 2:

| Ingredient | Amount (mg) | % of Total Comp |
|---|---|---|
| Vitamin D (as | 0.015 | 0.001% |
| cholecalciferol) | | |
| Calcium (as calcium | 550 | 20.754% |
| carbonate CaCO₃) | | |
| Ginger Root Powder | 800 | 30.188% |
| Olive Leaf Powder | 500 | 18.868% |
| Cinnamon Bark | 500 | 18.868% |
| Powder | | |
| Garlic Bulb Extract | 300 | 11.321% |
| Total: | 2650.015 | 99.999% |

Formula 3:

| Ingredient | Amount (mg) | % of Total Comp |
|---|---|---|
| Vitamin D (as | 0.02 | 0.001% |
| cholecalciferol) | | |
| Calcium (as calcium | 550 | 20.754% |
| carbonate CaCO₃) | | |
| Ginger Root Powder | 750 | 28.301% |
| Olive Leaf Powder | 550 | 20.754% |
| Cinnamon Bark | 500 | 18.868% |
| Powder | | |
| Garlic Bulb Extract | 300 | 11.321% |
| Total: | 2650.02 | 99.999% |

Formula 4:

| Ingredient | Amount (mg) | % of Total Comp |
|---|---|---|
| Vitamin D (as | 0.025 | 0.001% |
| cholecalciferol) | | |
| Calcium (as calcium | 550 | 20.754% |
| carbonate CaCO₃) | | |
| Ginger Root Powder | 750 | 28.301% |
| Olive Leaf Powder | 500 | 18.868% |
| Cinnamon Bark | 550 | 20.754% |
| Powder | | |
| Garlic Bulb Extract | 300 | 11.321% |
| Total: | 2650.025 | 99.999% |

Formula 5:

| Ingredient | Amount (mg) | % of Total Comp |
|---|---|---|
| Vitamin D (as | 0.03 | 0.001% |
| cholecalciferol) | | |
| Calcium (as calcium | 550 | 20.754% |
| carbonate CaCO₃) | | |
| Ginger Root Powder | 750 | 28.301% |
| Olive Leaf Powder | 500 | 18.868% |
| Cinnamon Bark Powder | 500 | 18.868% |
| Garlic Bulb Extract | 350 | 13.207% |
| Total: | 2650.03 | 99.999% |

Formula 6:

| Ingredient | Amount (mg) | % of Total Comp |
|---|---|---|
| Vitamin D (as | 0.035 | 0.001% |
| cholecalciferol) | | |
| Calcium (as calcium | 500 | 18.868% |
| carbonate CaCO₃) | | |
| Ginger Root Powder | 850 | 32.075% |
| Olive Leaf Powder | 500 | 18.868% |
| Cinnamon Bark Powder | 500 | 18.868% |
| Garlic Bulb Extract | 300 | 11.321% |
| Total: | 2650.035 | 99.999% |

Formula 7:

| Ingredient | Amount (mg) | % of Total Comp |
|---|---|---|
| Vitamin D (as | 0.04 | 0.002% |
| cholecalciferol) | | |
| Calcium (as calcium | 500 | 18.868% |
| carbonate CaCO₃) | | |
| Ginger Root Powder | 750 | 28.301% |
| Olive Leaf Powder | 600 | 22.641% |
| Cinnamon Bark Powder | 500 | 18.868% |
| Garlic Bulb Extract | 300 | 11.321% |
| Total: | 2650.04 | 100.000% |

Formula 8:

| Ingredient | Amount (mg) | % of Total Comp |
|---|---|---|
| Vitamin D (as | 0.045 | 0.002% |
| cholecalciferol) | | |
| Calcium (as calcium | 500 | 18.868% |
| carbonate CaCO₃) | | |
| Ginger Root Powder | 750 | 28.301% |
| Olive Leaf Powder | 500 | 18.868% |
| Cinnamon Bark Powder | 600 | 22.641% |
| Garlic Bulb Extract | 300 | |
| Total: | | 100% |

Formula 9:

| Ingredient | Amount (mg) | % of Total Comp |
|---|---|---|
| Vitamin D (as | 0.055 | 0.002% |
| cholecalciferol) | | |
| Calcium (as calcium | 500 | 18.868% |
| carbonate CaCO₃) | | |
| Ginger Root Powder | 750 | 28.301% |
| Olive Leaf Powder | 500 | 18.868% |
| Cinnamon Bark Powder | 500 | 18.868% |
| Garlic Bulb Extract | 400 | 15.094% |
| Total: | 2650.055 | 100.000% |

Formula 10:

| Ingredient | Amount (mg) | % of Total Comp |
|---|---|---|
| Vitamin D (as | 0.06 | 0.002% |
| cholecalciferol) | | |
| Calcium (as calcium | 600 | 22.641% |
| carbonate CaCO₃) | | |
| Ginger Root Powder | 700 | 26.415% |
| Olive Leaf Powder | 550 | 20.754% |
| Cinnamon Bark Powder | 500 | 18.868% |
| Garlic Bulb Extract | 300 | 11.321% |
| Total: | 2650.06 | 100.000% |

Formula 11:

| Ingredient | Amount (mg) | % of Total Comp |
|---|---|---|
| Vitamin D (as | 0.065 | 0.002% |
| cholecalciferol) | | |
| Calcium (as calcium | 600 | 22.641% |
| carbonate CaCO₃) | | |
| Ginger Root Powder | 700 | 26.415% |
| Olive Leaf Powder | 500 | 18.868% |
| Cinnamon Bark Powder | 550 | 20.754% |
| Garlic Bulb Extract | 300 | 11.321% |
| Total: | 2650.065 | 100.001% |

Formula 12:

| Ingredient | Amount (mg) | % of Total Comp |
|---|---|---|
| Vitamin D (as | 0.07 | 0.003% |
| cholecalciferol) | | |
| Calcium (as calcium | 600 | 22.641% |
| carbonate CaCO₃) | | |
| Ginger Root Powder | 700 | 26.415% |
| Olive Leaf Powder | 500 | 18.868% |
| Cinnamon Bark Powder | 500 | 18.868% |
| Garlic Bulb Extract | 350 | 13.207% |
| Total: | 2650.07 | 100.001% |

Formula 13:

| Ingredient | Amount (mg) | % of Total Comp |
|---|---|---|
| Vitamin D (as | 0.075 | 0.003% |
| cholecalciferol) | | |
| Calcium (as calcium | 600 | 22.641% |
| carbonate CaCO₃) | | |
| Ginger Root Powder | 650 | 24.528% |
| Olive Leaf Powder | 600 | 22.641% |
| Cinnamon Bark Powder | 500 | 18.868% |
| Garlic Bulb Extract | 300 | 11.321% |
| Total: | 2650.075 | 100.001% |

Formula 14:

| Ingredient | Amount (mg) | % of Total Comp |
|---|---|---|
| Vitamin D (as | 0.08 | 0.003% |
| cholecalciferol) | | |
| Calcium (as calcium | 600 | 22.641% |
| carbonate CaCO₃) | | |
| Ginger Root Powder | 650 | 24.528% |
| Olive Leaf Powder | 500 | 18.868% |
| Cinnamon Bark Powder | 600 | 22.641% |
| Garlic Bulb Extract | 300 | 11.321% |
| Total: | 2650.08 | 100.001% |

Formula 15:

| Ingredient | Amount (mg) | % of Total Comp |
|---|---|---|
| Vitamin D (as | 0.085 | 0.003% |
| cholecalciferol) | | |
| Calcium (as calcium | 600 | 22.641% |
| carbonate CaCO₃) | | |
| Ginger Root Powder | 650 | 24.528% |
| Olive Leaf Powder | 500 | 18.868% |
| Cinnamon Bark Powder | 500 | 18.868% |
| Garlic Bulb Extract | 400 | 15.094% |
| Total: | 2650.085 | 100.001% |

Formula 16:

| Ingredient | Amount (mg) | % of Total Comp |
|---|---|---|
| Vitamin D (as | 0.09 | 0.003% |
| cholecalciferol) | | |
| Calcium (as calcium | 600 | 22.641% |
| carbonate CaCO₃) | | |
| Ginger Root Powder | 600 | 22.641% |
| Olive Leaf Powder | 650 | 24.528% |
| Cinnamon Bark Powder | 500 | 18.868% |
| Garlic Bulb Extract | 300 | 11.321% |
| Total: | 2650.09 | 100.002% |

Formula 17:

| Ingredient | Amount (mg) | % of Total Comp |
|---|---|---|
| Vitamin D (as | 0.095 | 0.004% |
| cholecalciferol) | | |
| Calcium (as calcium | 600 | 22.641% |
| carbonate CaCO₃) | | |
| Ginger Root Powder | 600 | 22.641% |
| Olive Leaf Powder | 500 | 18.868% |
| Cinnamon Bark Powder | 650 | 24.528% |
| Garlic Bulb Extract | 300 | 11.321% |
| Total: | 2650.095 | 100.002% |

Formula 18:

| Ingredient | Amount (mg) | % of Total Comp |
|---|---|---|
| Vitamin D (as | 0.1 | 0.004% |
| cholecalciferol) | | |
| Calcium (as calcium | 600 | 22.641% |
| carbonate CaCO₃) | | |
| Ginger Root Powder | 600 | 22.641% |
| Olive Leaf Powder | 500 | 18.868% |
| Cinnamon Bark Powder | 500 | 18.868% |
| Garlic Bulb Extract | 450 | 16.981% |
| Total: | 2650.1 | 100.002% |

Formula 19:

| Ingredient | Amount (mg) | % of Total Comp |
|---|---|---|
| Vitamin D (as | 0.05 | 0.002% |
| cholecalciferol) | | |
| Calcium (as calcium | 600 | 22.641% |
| carbonate CaCO₃) | | |
| Ginger Root Powder | 550 | 20.754% |
| Olive Leaf Powder | 700 | 26.415% |
| Cinnamon Bark Powder | 500 | 18.868% |
| Garlic Bulb Extract | 300 | 11.321% |
| Total: | | 100% |

Formula 20:

| Ingredient | Amount (mg) | % of Total Comp |
|---|---|---|
| Vitamin D (as | 0.05 | 0.002% |
| cholecalciferol) | | |
| Calcium (as calcium | 600 | 22.641% |
| carbonate CaCO₃) | | |
| Ginger Root Powder | 550 | 20.754% |
| Olive Leaf Powder | 500 | 18.868% |
| Cinnamon Bark Powder | 700 | 26.415% |
| Garlic Bulb Extract | 300 | 11.321% |
| Total: | 2650.05 | 100.000% |

Formula 21:

| Ingredient | Amount (mg | % of Total Comp |
|---|---|---|
| Vitamin D (as | 0.05 | 0.002% |
| cholecalciferol) | | |
| Calcium (as calcium | 600 | 22.641% |
| carbonate CaCO₃) | | |
| Ginger Root Powder | 550 | 20.754% |
| Olive Leaf Powder | 500 | 18.868% |
| Cinnamon Bark Powder | 500 | 18.868% |
| Garlic Bulb Extract | 500 | 18.868% |
| Total: | 2650.05 | 100.000% |

Formula 22:

| Ingredient | Amount (mg) | % of Total Comp |
|---|---|---|
| Vitamin D (as | 0.05 | 0.002% |
| cholecalciferol) | | |
| Calcium (as calcium | 600 | 22.641% |
| carbonate CaCO₃) | | |
| Ginger Root Powder | 500 | 18.868% |
| Olive Leaf Powder | 750 | 28.301% |
| Cinnamon Bark Powder | 500 | 18.868% |
| Garlic Bulb Extract | 300 | 11.321% |
| Total: | 2650.05 | 100.000% |

Formula 23:

| Ingredient | Amount (mg) | % of Total Comp |
|---|---|---|
| Vitamin D (as | 0.05 | 0.002% |
| cholecalciferol) | | |
| Calcium (as calcium | 600 | 22.641% |
| carbonate CaCO₃) | | |
| Ginger Root Powder | 500 | 18.868% |
| Olive Leaf Powder | 500 | 18.868% |
| Cinnamon Bark Powder | 750 | 28.301% |
| Garlic Bulb Extract | 300 | 11.321% |
| Total: | 2650.05 | 100.000% |

Formula 24:

| Ingredient | Amount (mg) | % of Total Comp |
|---|---|---|
| Vitamin D (as | 0.05 | 0.002% |
| cholecalciferol) | | |
| Calcium (as calcium | 600 | 22.641% |
| carbonate CaCO₃) | | |
| Ginger Root Powder | 500 | 18.868% |
| Olive Leaf Powder | 500 | 18.868% |
| Cinnamon Bark Powder | 500 | 18.868% |
| Garlic Bulb Extract | 550 | 20.754% |
| Total: | 2650.05 | 100.000% |

Each formula specifies a particular ingredient composition and corresponding amount and weight percentage of each ingredient out of a total daily serving active ingredient mass of approximately 2650 mg. In embodiments, the dietary supplement composition is contained in capsules, which can be gelatin capsules, for example made of bovine gelatin of a standard size. For example, the standard size can be selected from size 000, 00E, 00, 0E or 0. An amount of the composition containing a unit fraction of the daily serving amount (such as the entire daily serving amount, one half, one third, one fourth, or one fifth of the amount, etc.) is contained in each capsule, so that the daily serving amount is contained in one or a plural whole number of the capsules. In one embodiment, the dietary supplement composition of Formula 1 is contained in five size 00 capsules.

A method to improve and maintain a person's immunity and wellness using the dietary supplement compositions includes administering one or more capsules to a person daily for a plurality of days, and the person taking the one or more capsules orally, preferably with food. The capsules may be self-administered by the person taking them or administered by the person's guardian or caretaker. According to an embodiment, if a daily serving is contained in five capsules, a person could take two capsules with breakfast, two with lunch, and one with dinner; take three with breakfast and two with dinner; take two with breakfast and three with dinner, or otherwise distribute the capsules among the day's meals.

In accordance with each formula set forth above, the appropriate ingredients in the correct proportions are admixed to provide a dietary supplement composition with specific desired effects, while minimizing symptoms of potential side effects. The mixture is formed into, for example, multiple size 00 capsules or pills. Servings of the dietary supplement compositions are taken daily for specific improvement and maintenance of health and wellness.

It will be appreciated that many additional modifications and variations of the present disclosure are possible in light of the above teachings. Thus, it is to be understood that, within the scope of the appended claims, the disclosure may be practiced otherwise than is specifically described above. For instance, the composition may be administered in the form of, for example, a pill, a tablet, a pellet, a bead, a syrup, a liquid, a dissolvable powder, a particle, a solution, a spray, a paste, an ointment, a gel and any combinations thereof.

The foregoing description of the disclosure has been presented for purposes of illustration and description, and is not intended to be exhaustive or to limit the disclosure to the precise form disclosed. The description was selected to best explain the principles of the present teachings and practical application of these principles to enable others skilled in the art to best utilize the disclosure in various embodiments and various modifications as are suited to the particular use contemplated. It should be recognized that the words “a” or “an” are intended to include both the singular and the plural. Conversely, any reference to plural elements shall, where appropriate, include the singular.

It is intended that the scope of the disclosure not be limited by the specification, but be defined by the claims set forth below. In addition, although narrow claims may be presented below, it should be recognized that the scope of this invention is much broader than presented by the claim(s). It is intended that broader claims will be submitted in one or more applications that claim the benefit of priority from this application. Insofar as the description above and the accompanying drawings disclose additional subject matter that is not within the scope of the claim or claims below, the additional inventions are not dedicated to the public and the right to file one or more applications to claim such additional inventions is reserved.

What is claimed is:

1. A dietary supplement composition for promoting human immunity and wellness, the composition comprising:
a daily serving of each of vitamin D, calcium, ginger root powder, olive leaf powder, cinnamon bark powder, and garlic bulb extract.

2. The composition of claim 1 wherein the daily servings comprise:
an amount of Vitamin D from 600 to 4,000 international units (IU);
an amount of calcium from 500 to 600 mg;
an amount of ginger root powder from 500 to 850 mg;
an amount of olive leaf powder from 500 to 750 mg;
an amount of cinnamon bark powder from 500 to 750 mg; and
an amount of garlic bulb extract from 300 to 550 mg.

3. The composition of claim 2 wherein the daily servings comprise:
2,000 IU of Vitamin D;
600 mg of calcium;
750 mg of ginger root powder;
500 mg of olive leaf powder;
500 mg of cinnamon bark powder; and
300 mg of garlic bulb extract.

4. The composition of claim 2 wherein the daily servings comprise:
600 IU of Vitamin D;
550 mg of calcium;
800 mg of ginger root powder;
500 mg of olive leaf powder;
500 mg of cinnamon bark powder; and
300 mg of garlic bulb extract.

5. The composition of claim 2 wherein the daily servings comprise:
800 IU of Vitamin D;
550 mg of calcium;
750 mg of ginger root powder;
550 mg of olive leaf powder;
500 mg of cinnamon bark powder; and
300 mg of garlic bulb extract.

6. The composition of claim 2 wherein the daily servings comprise:
1000 IU of Vitamin D;
550 mg of calcium;
750 mg of ginger root powder;
500 mg of olive leaf powder;
550 mg of cinnamon bark powder; and
300 mg of garlic bulb extract.

7. The composition of claim 2 wherein the daily servings comprise:
1200 IU of Vitamin D;
550 mg of calcium;
750 mg of ginger root powder;
500 mg of olive leaf powder;
500 mg of cinnamon bark powder; and
350 mg of garlic bulb extract.

8. A method for promoting human immunity and wellness comprising:
preparing a daily serving of a dietary supplement composition including therapeutically effective daily serving amounts of each of vitamin D, calcium, ginger root powder, olive leaf powder, cinnamon bark powder, and garlic bulb extract;
a person taking the daily serving of the dietary supplement composition daily for a plurality of days.

9. The method of claim 8 wherein said preparing the daily serving of the dietary supplement composition comprises filling each of one or more capsules with an amount of the dietary supplement composition equal to a unit fraction of the daily serving, said taking the daily serving comprising the person taking a whole number of the one or more capsules equal to the inverse of said unit fraction.

10. The method of claim 9 wherein said preparing the daily serving of the dietary supplement composition comprises filling each of five capsules with one fifth of the daily serving, said taking the daily serving comprising the person taking the five capsules.

11. The method of claim 9 wherein the one or more capsules are gelatin capsules.

12. The method of claim 11, wherein the daily serving of the dietary composition further comprises at least one excipient.

13. The method of claim 12 wherein the at least one excipient is selected from rice flour and a stearic acid salt.

14. The method of claim 13 wherein the at least one excipient includes magnesium stearate.

15. The method of claim 14 wherein the magnesium stearate is vegetable magnesium stearate.

16. The method of claim 8 wherein the daily serving amounts comprise:
an amount of Vitamin D from 600 to 4,000 international units (IU);
an amount of calcium from 500 to 600 mg;
an amount of ginger root powder from 500 to 850 mg;
an amount of olive leaf powder from 500 to 750 mg;
an amount of cinnamon bark powder from 500 to 750 mg; and
an amount of garlic bulb extract from 300 to 550 mg.

17. The method of claim 16 wherein the daily serving amounts comprise:
2,000 IU of Vitamin D;
600 mg of calcium;
750 mg of ginger root powder;
500 mg of olive leaf powder;
500 mg of cinnamon bark powder; and
300 mg of garlic bulb extract.

18. The method of claim 16 wherein the daily serving amounts comprise:
600 IU of Vitamin D;
550 mg of calcium;
800 mg of ginger root powder;
500 mg of olive leaf powder;
500 mg of cinnamon bark powder; and
300 mg of garlic bulb extract.

19. The method of claim 16 wherein the daily serving amounts comprise:
800 IU of Vitamin D;
550 mg of calcium;
750 mg of ginger root powder;
550 mg of olive leaf powder;
500 mg of cinnamon bark powder; and
300 mg of garlic bulb extract.

20. The method of claim 16 wherein the daily serving amounts comprise:
1000 IU of Vitamin D;
550 mg of calcium;
750 mg of ginger root powder;
500 mg of olive leaf powder;
550 mg of cinnamon bark powder; and
300 mg of garlic bulb extract.
